(19) United States
(12) Patent Application Publication
(10) Pub. No.: US 2019/0111101 A1
(43) Pub. Date: Apr. 18, 2019

(54) USES OF SHELL-FLOWER ESSENTIAL OIL

(71) Applicant: TCI Co., Ltd., Taipei (TW)

(72) Inventor: Yung-Hsiang LIN, Taipei (TW)

(21) Appl. No.: 16/158,461

(22) Filed: Oct. 12, 2018

Related U.S. Application Data
(60) Provisional application No. 62/573,929, filed on Oct. 18, 2017.

(30) Foreign Application Priority Data
Sep. 21, 2018 (TW) 107133383

(51) Int. Cl. A61K 36/9062; A61P 3/00; A61P 39/06; A61P 25/28

(52) U.S. Cl. CPC A61K 36/9062; A61P 25/28; A61P 39/06; A61P 3/00

(57) ABSTRACT

A method for anti-aging, promoting metabolism, and/or assisting in reducing production of free radicals is provided, wherein the method comprises administering to a subject in need an effective amount of shell-flower essential oil. A method for at least one of preventing cancer, treating cancer, and inhibiting degenerative disease caused by mitochondrial aging is also provided, wherein the method comprises administering to a subject in need an effective amount of shell-flower essential oil.

### CROSS REFERENCE TO RELATED APPLICATIONS

This application claims priority to U.S. Provisional Application Ser. No. 62/573,929 filed on Oct. 18, 2017, and to Taiwan Patent Application No. 107133383 filed on Sep. 21, 2018, the disclosures of which are incorporated herein in their entirety by reference.

### FIELD OF THE INVENTION

The present invention relates to the use of shell-flower (Alpinia zerumbet) essential oil, including the use of shell-flower essential oil in delaying aging, promoting metabolism, and/or assisting in reducing production of free radicals. The present invention also relates to the use of shell-flower essential oil in anti-aging, preventing gastric cancer, and/or treating gastric cancer. The present invention also relates to the use of shell-flower essential oil in inhibiting degenerative disease caused by mitochondrial aging.

### BACKGROUND OF THE INVENTION

Mitochondria are the primary organelles responsible for generating energy in cells. In addition to supplying energy to cells, mitochondria are involved in cell differentiation, cell signal transduction, and apoptosis, and are capable of regulating cell growth and cell cycle. When mitochondrial DNA mutates, the number of mitochondria decreases, or the function of mitochondria declines, those may cause cells being unable to generate sufficient energy to supply the normal functions of body, tissues and organs, and thus, lead to many diseases. The tendency of functional impairment due to mitochondrial problems is particularly significant in the organs with high energy consumption (e.g., brain, skeletal muscles, cardiac muscles and liver). If the functions of the organs in a subject gradually decline due to failing to access sufficient energy and the subject is not treated in time, eventually the subject will die because of the cease of physiological functions.

Free radicals exist in various parts of the human body and are very active and unstable. Under the normal situation, the body has enough ability to remove or neutralize the free radicals, and thus, can avoid any damage to the body caused by free radicals. However, factors such as radiation, pollution, smoking and agricultural chemicals in the environment and a lifestyle with irregularity work and rest all may lead to the production of more free radicals and cause damages to normal cells, and thus result in the impairments in the body tissues and organs as well as the occurrences of aging phenomena (e.g., skin aging, fatigue, memory decline, slow metabolism, organ degeneration) and diseases.

Inventors of the present invention incidentally discovered that shell-flower essential oil can increase the expressions of GPX1 gene, CCT2 gene, CCT6A gene, CCT8 gene, and/or Atg1 gene of a subject, and thus, is effective in anti-oxidation, inhibiting cellular senescence and enhancing mitochondrial activity. Hence, shell-flower essential oil can be used for delaying aging, promoting metabolism, assisting in reducing production of free radicals, anti-aging, preventing cancer, treating cancer, and/or inhibiting degenerative disease caused by mitochondrial aging.

### SUMMARY OF THE INVENTION

An objective of the present invention is to provide a use of shell-flower essential oil in at least one of delaying aging, promoting metabolism, and assisting in reducing production of free radicals. Preferably, the shell-flower essential oil is taken through oral, inhalation, or transdermal route.

Another objective of the present invention is to provide a use of shell-flower essential oil in the manufacture of a pharmaceutical composition for anti-aging, preventing cancer, and/or treating cancer. Preferably, the cancer is gastric cancer. Preferably, the pharmaceutical composition is for delaying degeneration of body functions and is provided in a form for oral administration, inhalation administration, or transdermal administration.

Still another objective of the present invention is to provide a use of shell-flower essential oil in the manufacture of a pharmaceutical composition for inhibiting degenerative disease caused by mitochondrial aging. Preferably, the pharmaceutical composition is for inhibiting neurodegenerative disease caused by mitochondrial aging and is provided in a form for oral administration, inhalation administration, or transdermal administration.

Yet another objective of the present invention is to provide a use of shell-flower essential oil in the manufacture of a pharmaceutical composition for increasing the expressions of at least one of GPX1 gene, CCT2 gene, CCT6A gene, CCT8 gene, and Atg1 gene. Preferably, the pharmaceutical composition is provided in a form for oral administration, inhalation administration, or transdermal administration.

Yet another objective of the present invention is to provide a care product composition for delaying aging, promoting metabolism, and/or assisting in reducing production of free radicals. The care product composition comprises an effective amount of shell-flower essential oil. Preferably, the care product composition is provided in a form for oral administration, inhalation administration, or transdermal administration.

Yet another objective of the present invention is to provide a pharmaceutical composition for anti-aging, preventing cancer, and/or treating cancer. The pharmaceutical composition comprises an effective amount of shell-flower essential oil. Preferably, the cancer is gastric cancer. Preferably, the pharmaceutical composition is for delaying degeneration of body functions and is provided in a form for oral administration, inhalation administration, or transdermal administration.

Yet another objective of the present invention is to provide a pharmaceutical composition for inhibiting degenerative disease caused by mitochondrial aging. The pharmaceutical composition comprises an effective amount of shell-flower essential oil. Preferably, the pharmaceutical composition is for inhibiting neurodegenerative disease caused by mitochondrial aging and is provided in a form for oral administration, inhalation administration, or transdermal administration.

Yet another objective of the present invention is to provide a pharmaceutical composition for increasing the expressions of GPX1 gene, CCT2 gene, CCT6A gene, CCT8 gene, and/or Atg1 gene. The pharmaceutical composition comprises an effective amount of shell-flower essential oil. Preferably, the pharmaceutical composition is provided in a form for oral administration, inhalation administration, or transdermal administration.

Yet another objective of the present invention is to provide a method for at least one of anti-aging, promoting metabolism, and assisting in reducing production of free radicals, comprising administering to a subject in need an effective amount of shell-flower essential oil. In the method of the present invention, the shell-flower essential can be administered as the care product composition or the pharmaceutical composition described above. Preferably, the method is for delaying degeneration of body functions.

Yet another objective of the present invention is to provide a method for at least one of preventing cancer, treating cancer, and inhibiting degenerative disease caused by mitochondrial aging, comprising administering to a subject in need an effective amount of shell-flower essential oil. In the method of the present invention, the shell-flower essential oil can be administered as the pharmaceutical composition described above. Preferably, the cancer is gastric cancer. Preferably, the method is for inhibiting neurodegenerative disease caused by mitochondrial aging.

Yet another objective of the present invention is to provide a method for increasing the expressions of GPX1 gene, CCT2 gene, CCT6A gene, CCT8 gene, and/or Atg1 gene, comprising administering to a subject in need an effective amount of shell-flower essential oil. In the method of the present invention, the shell-flower essential oil can be administered as the pharmaceutical composition described above.

The detailed technology and preferred embodiments implemented for the present invention are described in the following paragraphs accompanying the appended drawings for people skilled in this field to well appreciate the features of the claimed invention.

### BRIEF DESCRIPTION OF THE DRAWINGS

FIG. 1 shows the influence of shell-flower essential oil on the expression of GPX1 gene in peripheral blood mononuclear cells (PBMCs), wherein the result of “W0” group refers to the expression level before the administration of shell-flower essential oil, and the results of “W1” and “W2” groups refer to the expression levels after the continuous administration of shell-flower essential oil for one week and two weeks, respectively (** represents the result is significantly different from that of the “W0” group, p<0.01)

FIG. 2 shows the influence of shell-flower essential oil on the expression of CCT2 gene in PBMCs of the “W0”, “W1” and “W2” groups (* represents the result is significantly different from that of the “W0” group, p<0.05);

FIG. 3 shows the influence of shell-flower essential oil on the expression of CCT6A gene in PBMCs of the “W0”, “W1” and “W2” groups (* represents the result is significantly different from that of the “W0” group, p<0.05)

FIG. 4 shows the influence of shell-flower essential oil on the expression of CCT8 gene in PBMCs of the “W0”, “W1” and “W2” groups (* represents the result is significantly different from that of the “W0” group, p<0.05); and

FIG. 5 shows the influence of shell-flower essential oil on the expression of Atg1 gene in PBMCs of the “W0”, “W1” and “W2” groups (* represents the result is significantly different from that of the “W0” group, p<0.05).

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification. In addition, unless otherwise indicated herein, the expressions “a,” “an,” “the,” or the like recited in the specification of the present invention (especially in the claims) are intended to include both the singular and plural forms. The term “treat” or “treating” used in this specification should not be construed as treating a subject until the subject completely recovered, but should include maintaining the progression or symptoms of the diseases in a substantially static state, increasing the recovery rate of a subject, alleviating the severity of a particular condition of illness, or increasing the life quality of patients. The term “prevent” or “preventing” recited in this specification refers to inhibiting or avoiding a particular condition of illness from breaking out, maintaining good health in a sensitive subject, or establishing the ability of a sensitive subject to tolerate diseases. The term “subject” recited in this specification refers to a mammalian, including human and non-human animals.

It was revealed by researches that an increment in the expression level of GPX1 gene is beneficial to the removal of cellular peroxides and the reduction of free radical production, and thus, is effective in enhancing the antioxidant capability of cells. Therefore, if the expression of GPX1 gene can be increased effectively, the effects of anti-oxidation and/or assisting in reducing the production of free radicals can be provided.

Researches also revealed that the low expression or deletion of GPX1 gene is related to the occurrence of cancer such as gastric cancer, and these can be noted in “Prognostic significance of glutathione peroxidase 1 (GPX1) down-regulation and correlation with aberrant promoter methylation in human gastric cancer. Anticancer Research. 32: 3169-3176 (2012)”, which is entirely incorporated hereinto by reference. Therefore, if the expression of GPX1 gene can be increased, the effects of preventing cancer and/or treating cancer can be provided, particularly the effects of preventing gastric cancer and/or treating gastric cancer.

It was revealed by researches that an increment in the expression levels of CCT2, CCT6A and CCT8 genes is beneficial to the rejuvenation of mature cells, thereby providing the effects of anti-aging and extending lifespan of organisms, and these can be noted in “Somatic increase of CCT8 mimics proteostasis of human pluripotent stem cells and extends C. elegans lifespan. Nature Communications. 7: 13649 (2016)”, which is entirely incorporated hereinto by reference. Therefore, if the expressions of CCT2, CCT6A and CCT8 genes can be increased, the effects of delaying aging and/or anti-aging can be provided.

In addition, it is known that the mutation of mitochondrial DNA may cause many diseases associated with mitochondria and is closely related to aging and the occurrences of aging diseases. Researches have shown that an increment in the expression level of Atg1 gene is beneficial to the removal of mutant mitochondrial DNA, and this can be noted in “Mitochondrial DNA mutations in disease and aging. J. Cell Biol. 193(5): 809-818 (2011)” and “Selective removal of deletion-bearing mitochondrial DNA in heteroplasmic Drosophila. Nature Communications. 7: 13100 (2016)”, which are entirely incorporated hereinto by reference. Therefore, if the expression of Atg1 gene can be increased, the mutant motichondrial DNA can be effectively removed, thereby providing the following effects: anti-aging, promoting metabolism, delaying degeneration of body functions, and/or inhibiting degenerative disease caused by mitochondrial aging, particularly inhibiting neurodegenerative disease caused by mitochondrial aging.

Essential oil is an oil product containing aromatic compound(s) and is extracted from plants. Specifically, essential oil can be provided by extracting the flowers, leaves, roots, seeds, peels, fruits and/or stems of plant(s) by such as distillation, expeller press, solvent extraction, enfleurage, and supercritical fluid extraction (SFE). Depending on the species and parts of plant(s) from which the essential oil is produced, the essential oil may have different effects and can be used in different forms in daily life or in the pharmaceutical industry as well.

Inventors of the present invention incidentally discovered that shell-flower essential oil can effectively increase the expressions of GPX1, CCT2, CCT6A, CCT8, and Atg1 genes. Therefore, the present invention relates to the use of shell-flower essential oil, including: using shell-flower essential oil in delaying aging, promoting metabolism, and/or assisting in reducing production of free radicals, using shell-flower essential oil in manufacturing a pharmaceutical composition, providing a care product composition or a pharmaceutical composition comprising an effective amount of shell-flower essential oil, and providing a method of administering to a subject in need an effective amount of the aforementioned care product composition or pharmaceutical composition. The care product composition provided in accordance with the present invention is for delaying aging, promoting metabolism, and/or assisting in reducing production of free radicals. The pharmaceutical composition provided in accordance with the present invention is for anti-aging, preventing cancer, and/or treating cancer, particularly for delaying degeneration of body functions. The pharmaceutical composition is also for inhibiting degenerative disease caused by mitochondrial aging, particularly for inhibiting neurodegenerative disease caused by mitochondrial aging. In addition, the pharmaceutical composition and the method provided in accordance with the present invention are also for increasing the expressions of GPX1 gene, CCT2 gene, CCT6A gene, CCT8 gene, and/or Atg1 gene.

The shell-flower essential oil adopted in accordance with the present invention could be obtained from any suitable source. For example, the shell-flower essential oil could be obtained by extracting shell flower (e.g., extracting the leaves, roots or stems of shell flower via distillation), and could also be purchased from the market.

Depending on the desired purpose(s), the care product composition provided in accordance with the present invention could be administered to a subject in need systemically or topically, and could be provided in any suitable form without particular limitations. For example, the care product composition could be provided in a form for oral administration, inhalation administration, or transdermal administration, but is not limited thereby. In addition, depending on the desired purpose(s), the pharmaceutical composition provided in accordance with the present invention could be administered to a subject in need systemically or topically, and could be provided in any suitable form without particular limitations. For example, the pharmaceutical composition could be provided in a form for oral administration, inhalation administration or transdermal administration, but is not limited thereby.

Depending on the form and purpose(s), a suitable carrier could be chosen and used to provide the care product composition or the pharmaceutical composition. Examples of the carrier include excipients, diluents, auxiliaries, stabilizers, absorption enhancers, disintegrating agent, hydrotropic agents, emulsifiers, antioxidants, adhesives, binders, tackifiers, dispersants, suspending agents, lubricants, hygroscopic agents, etc.

As a form for oral administration, the care product composition of the present invention could comprise any carrier that will not adversely affect the desired effects of the active ingredient (i.e., shell-flower essential oil). Examples of the suitable carrier include, but are not limited to, water, saline, dextrose, glycerol, ethanol or its analogs, oil (e.g., olive oil, castor oil, cottonseed oil, peanut oil, corn oil, germ oil), polyethylene glycol and combinations thereof. The care product composition could be provided by any suitable method in any suitable form for oral administration, such as in the form of a solution, syrup, a suspension, and could also be manufactured as a product such as a capsule, a beverage (e.g., a beauty beverage, a functional beverage, a health beverage), but is not limited thereby.

As a form for oral administration, the pharmaceutical composition of the present invention could comprise any pharmaceutically acceptable carrier that will not adversely affect the desired effects of the active ingredient (i.e., shell-flower essential oil). Examples of the suitable carrier include, but are not limited to, water, saline, dextrose, glycerol, ethanol or its analogs, oil (e.g., olive oil, castor oil, cottonseed oil, peanut oil, corn oil, germ oil), polyethylene glycol and combinations thereof. The pharmaceutical composition could be provided by any suitable method in any suitable form for oral administration, such as in the form of a capsule, a fluidextract, a solution, syrup, a suspension, a tincture, or an elixir, but is not limited thereby.

As a form for inhalation administration, the care product composition and the pharmaceutical composition of the present invention could optionally be aerosolized by any suitable approach to facilitate the entry of the care product composition or the pharmaceutical composition into the respiratory tract. For example, the care product composition and the pharmaceutical composition could be administered through a nebulizer, an aroma burner, an evaporative diffuser, an aroma lamp, an aroma diffuser, an essential oil candle, or an essential oil necklace, but is not limited thereby.

As a form for transdermal administration, the care product composition of the present invention could comprise any dermatologically acceptable carrier that will not adversely affect the desired effects of the active ingredient (i.e., shell-flower essential oil). Examples of the suitable carrier include, but are not limited to, water, mineral oil, propylene glycol, polyethylene oxide, liquid petrolatum, sorbitan monostearate, polysorbate 60. The care product composition could be provided by any suitable method in any suitable form for transdermal administration, such as in the form of an emulsion (e.g., a massage emulsion), a cream (e.g., a massage cream), a gel (e.g., a hydrogel), a solution (e.g., an essence, a lotion), a spray, but is not limited thereby.

As a form for transdermal administration, the pharmaceutical composition of the present invention could also comprise any pharmaceutically acceptable carrier that will not adversely affect the desired effects of the active ingredient (i.e., shell-flower essential oil). Examples of the suitable carrier include, but are not limited to, water, mineral oil, propylene glycol, polyethylene oxide, liquid petrolatum, sorbitan monostearate, polysorbate 60. The pharmaceutical composition could be provided by any suitable method in any suitable form for transdermal administration, such as in a form of an emulsion (e.g., a massage emulsion), a cream (e.g., a massage cream), an oily product (e.g., a massage oil), a gel (e.g., a hydrogel), a paste (e.g., a dispersing paste, an ointment), a douche, a spray, a patch (e.g., a microneedle patch), but is not limited thereby.

The pharmaceutical composition provided in accordance with the present invention could be administered to a subject in need systemically or topically, and could be delivered by various drug delivery systems (DDSs), such as oral drug delivery system, transdermal drug delivery system, inhalation drug delivery system, etc. For example, to enhance bioavailability, control drug release speed, target the lesion precisely and reduce side effects, the pharmaceutical composition could be delivered by a liposome, a microcapsule, nanoparticles, microneedles, but is not limited thereby.

Optionally, the care product composition and the pharmaceutical composition provided in accordance with the present invention could further comprise a suitable amount of additives, such as a flavoring agent, a toner, or a coloring agent for enhancing the palatability and the visual perception of the care product composition or the pharmaceutical composition, and/or a buffer, a conservative, a preservative, an antibacterial agent, or an antifungal agent for improving the stability and storability of the care product composition or the pharmaceutical composition. Optionally, the care product composition and the pharmaceutical composition could further comprise one or more other active ingredients, or to be used in combination with a medicament comprising one or more other active ingredients, to further enhance the effects of the care product composition and the pharmaceutical composition, or to increase the application flexibility and adaptability of the preparation thus provided, as long as the other active ingredients do not adversely affect the desired effects of the active ingredient of the present invention (i.e., shell-flower essential oil).

Depending on the needs, age, body weight and health conditions of the subject as well as the purpose(s), the care product composition and the pharmaceutical composition provided in accordance with the present invention could be administered at various administration frequencies, such as once a day, multiple times a day, once every few days, etc. In addition, the concentration of the shell-flower essential oil in the care product composition and the pharmaceutical composition could be adjusted depending on the requirements of practical application.

In the use of shell-flower essential oil in delaying aging, promoting metabolism, and/or assisting in reducing production of free radicals in accordance with the present invention, the shell-flower essential oil could be provided as a care product composition and the administration type, administration route, administration form, administration frequency and uses of the care product composition are all in line with the above descriptions.

As described above, the present invention also provides a method for delaying aging, promoting metabolism, and/or assisting in reducing production of free radicals, comprising administering to a subject in need an effective amount of shell-flower essential oil, wherein “a subject in need” refers to a subject having aging phenomena, with low metabolic efficiency, and/or with excessive production of free radicals. In this method, the shell-flower essential oil could be administered to the subject in need as a care product composition and the administration type, administration route, administration form, administration frequency and uses the care product composition are also all in line with the above descriptions.

The present invention also provides a method for anti-aging, preventing cancer, treating cancer, and/or inhibiting degenerative disease caused by mitochondrial aging, comprising administering to a subject in need an effective amount of shell-flower essential oil, wherein “a subject in need” refers to a subject having aging phenomena, suffering from cancer, with high-risk of cancer, and/or suffering from degenerative disease caused by mitochondrial aging. For example, the subject is the one with degeneration of body functions or suffering from neurodegenerative disease caused by mitochondrial aging. In this method, the shell-flower essential oil could be administered to the subject in need as a pharmaceutical composition and the administration type, administration route, administration form, administration frequency and uses of the pharmaceutical composition are also all in line with the above descriptions.

The present invention also provides a method for increasing the expressions of GPX1 gene, CCT2 gene, CCT6A gene, CCT8 gene, and/or Atg1 gene, comprising administering to a subject in need an effective amount of shell-flower essential oil, wherein “a subject in need” refers to a subject having the deletion, mutation, or low expression of GPX1 gene, CCT2 gene, CCT6A gene, CCT8 gene, and/or Atg1 gene. In this method, the shell-flower essential oil could be administered to the subject in need as a pharmaceutical composition and the administration type, administration route, administration form, administration frequency and uses of the pharmaceutical composition are also all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Example 1: Effects of Shell-Flower Essential Oil on Increasing the Expressions of GPX1 Gene, CCT2 Gene, CCT6A Gene, CCT8 Gene and Atg1 Gene

### (1-1) Collection of Blood Samples

The following two-week trial was conducted on six people (six subjects) to understand the effects of shell-flower essential oil on human body. During the trial, each subject wore an essential oil necklace containing shell-flower essential oil (purchased from Taiwan Specific Plant Essential Oil) for 8 hours every day, and the essential oil necklace was added with 20 μL of shell-flower essential oil at nine o'clock every morning to ensure that the amount of essential oil in the necklace was sufficient. And, 6 mL blood sample was collected from each subject prior to the trial (i.e., prior to starting wearing the essential oil necklace containing shell-flower essential oil; hereinafter referred to as “W0” group), after starting the trial for one week (i.e., wearing the essential oil necklace containing shell-flower essential oil eight hours a day and for one week; hereinafter referred to as “W1” group), and after starting the trial for two weeks (i.e., wearing the essential oil necklace containing shell-flower essential oil eight hours a day and for two weeks; hereinafter referred to as “W2” group), respectively, by using blood collection tubes that contained ethylenediaminetetraacetic acid (EDTA) (purchased from VACUETTE®, product number: 456036). The blood samples thus obtained were used for the following experiments and analysis.

### (1-2) Isolation of Peripheral Blood Mononuclear Cells (PBMCs)

The blood samples of “W0” group, “W 1” group and “W2” group obtained from Example (1-1) were individually subjected to the following steps to isolate PBMCs from the blood samples of each group:

1. Injecting each blood sample to a 15 mL centrifuge tube, and subjecting the centrifuge tube to a centrifugation at 300 g for 15 minutes (the centrifugation was stopped by natural deceleration);

2. Removing the upper blood plasma layer, taking out 2 mL of buffy coat from the remnant and putting the same to another 15 mL centrifuge tube, and then adding thereinto 2 mL of phosphate buffered saline (PBS) and mixing evenly to provide a diluted buffy coat;

3. Tilting another 15 mL centrifuge tube loaded with 3 mL of Ficoll-Plague Plus (purchased from Sigma company, product number: GE17-1440-03) for about 45 degrees, then slowly adding the diluted buffy coat provided by step 2 into the centrifuge tube and carefully returning the centrifuge tube back to the vertical situation, so that the buffy coat and Ficoll-Plague Plus were layered (not mixed), and then, subjecting the centrifuge tube to a centrifugation at 400 g for 40 minutes (the centrifugation was stopped by natural deceleration);

4. Removing the upper liquid layer, taking out 2 mL to 3 mL of the middle layer (i.e., mononuclear cell layer) and putting the same to a new 15 mL centrifuge tube, then, washing the cells with PBS three to five times, and then, subjecting the washed cells to a centrifugation at 300 g for 10 minutes; and

5. Removing the remaining PBS to provide the precipitate, i.e., the PBMCs of “W0” group, “W 1” group, or “W2” group.

### (1-3) Gene Expression Analysis

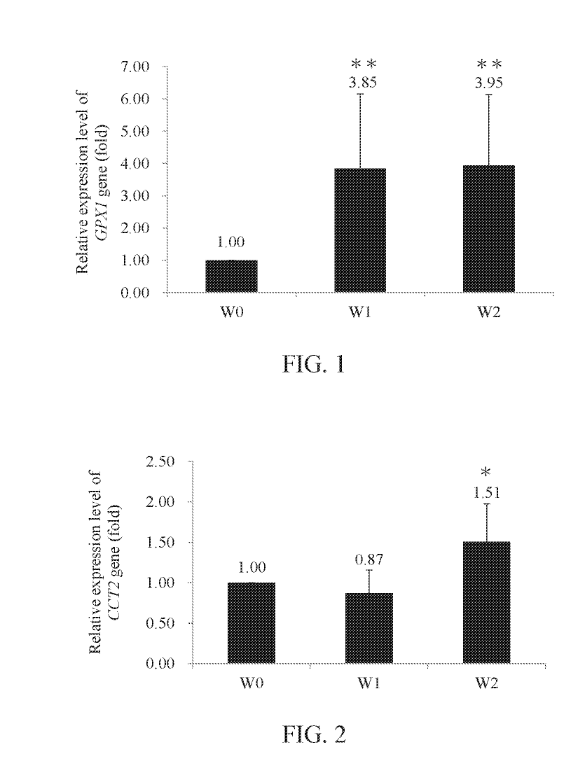

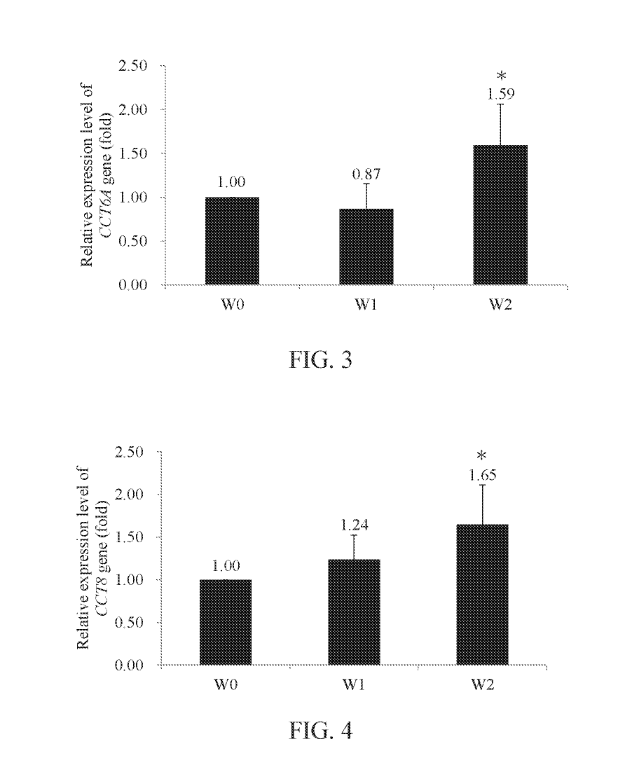

The PBMCs of “W0” group, “W1” group and “W2” group obtained from Example (1-2) were lysed by an RNA lysis buffer (RB buffer; purchased from Geneaid company). Thereafter, the PBMC lysate of each group was subjected to an RNA extraction with an RNA extraction kit (purchased from Geneaid company), and then the RNA was transcribed into cDNA with a reverse transcriptase (SuperScript® III Reverse Transcriptase; purchased from Invitrogen company). Then, the cDNA of each group was subjected to a quantitative polymerase chain reaction (qPCR) by using an ABI Step One Plus system and a KAPA SYBR FAST qPCR kit to determine the expression levels of GPX1, CCT2, CCT6A, CCT8 and Atg1 genes in the cells of each group. Finally, the data was analyzed by T.TEST of Excel (one tailed Student's t-test), and the analysis result of “W0” group was used as a basis (i.e., the gene expression of “W0” group was set as 1-fold) to calculate the relative gene expression level of “W1” group and that of “W2” group. The results are shown in FIGS. 1 to 5.

As shown in FIG. 1, as compared to “W0” group, the expression levels of GPX1 gene in the cells of “W 1” group and “W2” group significantly increased. These results indicate that shell-flower essential oil can effectively increase the expression level of GPX1 gene, and thus, can be used in anti-oxidation, assisting in reducing production of free radicals, preventing cancer, and/or treating cancer.

As shown in FIGS. 2 to 4, as compared to “W0” group, the expression levels of CCT2, CCT6A and CCT8 genes in the cells of “W2” group all significantly increased. These results indicate that shell-flower essential oil can effectively increase the expression levels of CCT2, CCT6A and CCT8 genes, and thus, can be used in delaying aging and/or anti-aging.

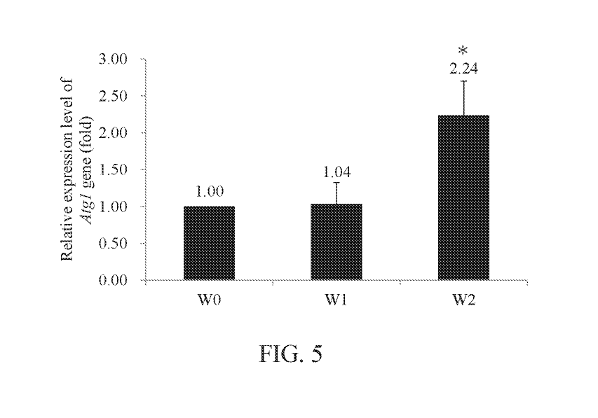

As shown in FIG. 5, as compared to “W0” group, the expression level of Atg1 gene in the cells of “W2” group significantly increased. This result indicates that shell-flower essential oil can effectively increase the expression level of Atg1 gene, and thus, can be used in anti-aging, promoting metabolism, delaying degeneration of body functions, and/or inhibiting degenerative disease caused by mitochondrial aging, particularly inhibiting neurodegenerative disease caused by mitochondrial aging.

What is claimed is:

1. A method for at least one of anti-aging, promoting metabolism, and assisting in reducing production of free radicals, comprising administering to a subject in need an effective amount of shell-flower essential oil.

2. The method as claimed in claim 1, which is for delaying degeneration of body functions.

3. The method as claimed in claim 1, wherein the shell-flower essential oil is administered to the subject by at least one of oral administration, inhalation administration, and transdermal administration.

4. The method as claimed in claim 2, wherein the shell-flower essential oil is administered to the subject by at least one of oral administration, inhalation administration, and transdermal administration.

5. A method for at least one of preventing cancer, treating cancer, and inhibiting degenerative disease caused by mitochondrial aging, comprising administering to a subject in need an effective amount of shell-flower essential oil.

6. The method as claimed in claim 5, wherein the cancer is gastric cancer.

7. The method as claimed in claim 5, which is for inhibiting neurodegenerative disease caused by mitochondrial aging.

8. The method as claimed in claim 5, wherein the shell-flower essential oil is administered to the subject by at least one of oral administration, inhalation administration, and transdermal administration.

9. The method as claimed in claim 6, wherein the shell-flower essential oil is administered to the subject by at least one of oral administration, inhalation administration, and transdermal administration.

10. The method as claimed in claim 7, wherein the shell-flower essential oil is administered to the subject by at least one of oral administration, inhalation administration, and transdermal administration.

11. A method for increasing the expressions of GPX1 gene, CCT2 gene, CCT6A gene, CCT8 gene, and/or Atg1 gene, comprising administering to a subject in need an effective amount of shell-flower essential oil.

12. The method as claimed in claim 11, wherein the shell-flower essential oil is administered to the subject by at least one of oral administration, inhalation administration, and transdermal administration.
